(19) United States
(12) Patent Application Publication
(10) Pub. No.: US 2025/0095820 A1
(43) Pub. Date: Mar. 20, 2025

(54) METHOD AND SYSTEM FOR GENERATING MASSAGE TRACKS

(71) Applicant: REALMAN INTELLIGENT TECHNOLOGY (BEIJING) CO., LTD., Beijing (CN)

(72) Inventors: Hui LAN, Beijing (CN); Ying ZHANG, Beijing (CN); Qinpeng DONG, Beijing (CN); Xulong ZHANG, Beijing (CN); Suibing ZHENG, Beijing (CN)

(73) Assignee: REALMAN INTELLIGENT TECHNOLOGY (BEIJING) CO., LTD., Beijing (CN)

(21) Appl. No.: 18/797,951

(22) Filed: Aug. 8, 2024

Related U.S. Application Data
(63) Continuation of application No. PCT/CN2023/140192, filed on Dec. 20, 2023.

(30) Foreign Application Priority Data
Sep. 18, 2023 (CN) 202311200597.X

(51) Int. Cl. G16H 20/30; A61H 99/00

(52) U.S. Cl. CPC G16H 20/30; A61H 99/00; A61H 2201/1659

(57) ABSTRACT

A method for generating massage tracks includes acquiring two-dimensional data of preliminary massage point positions according to a user-selected massage mode; acquiring point cloud data of a region to be massaged of a target object; acquiring the massage point position two-dimensional data of the target by proportion calculation according to the preliminary massage point position two-dimensional data and the point cloud data of the region to be massaged of the target object; acquiring three-dimensional massage point position data of the target according to the two-dimensional data of the massage point position of the target and the point cloud data of the region to be massaged of the target object; and generating a final massage track based on a preset massage track planning algorithm according to the three-dimensional massage point position data of the target. The present application may select a massage mode to improve individual adaptability.

### CROSS-REFERENCE TO RELATED APPLICATIONS

The present application is a continuation of International Patent Application No. PCT/CN2023/140192, filed on Dec. 20, 2023, which claims priority to Chinese Patent Application No. 202311200597.X, filed on Sep. 18, 2023. All of the aforementioned applications are incorporated herein by reference in their entireties.

### TECHNICAL FIELD

The present application relates to the field of robotic massage technology, and in particular to a method and a system for generating massage tracks.

### BACKGROUND

Massage tracks of massage equipment such as automatic massage devices such as massage chairs and massage beds are preset and can only provide a user with a massage according to a fixed massage track. The body type of client and the position where the target object lies will affect the massage effect, and the adaptability for different users is poor.

The physical interaction between robot and human is an emerging research field in recent years. Machine vision has developed rapidly in recent years. Many universities and research institutions combine the technology of machine vision with the massage robot to promote the further development of massage robot. Nowadays, with the development of computer technology, the modern intelligent robot technology has become more and more important. With the help of new technologies such as perception/Internet/cloud storage, the massage robot can help or replace the massager's action of conventional massage.

With the advent of massage robots, there is a tendency to plan massage tracks independently for different users. However, the tracks of the existing massage robot planning are mainly linear tracks with a single form, and the contour of the target object is poorly adapted. Based on the curve trajectory of Bessel curve planning, the spiral trajectory is limited by the characteristics of Bessel curve. The change of a massage point will affect the shape of the whole massage track, and the quantity of massage point positions is directly related to the order of Bessel curve. When the point increases, the calculation difficulty of Bessel curve will be increased, which is a challenge to the computing power of massage robot.

### SUMMARY

It is an object of the present application to provide a method and system for generating massage tracks that reduce the amount of calculation, improve the individual adaptability, and smooth the massage track.

In order to solve the above problems, the present application provides a method for generating massage tracks, comprising:
acquiring two-dimensional data of preliminary massage point positions according to a selected massage mode;
acquiring point cloud data of a region to be massaged of a target object;
determining a proportion coefficient according to the two-dimensional data of the preliminary massage point position and the point cloud data of the region to be massaged of the target object, and determining the two-dimensional data of the massage point position of the target based on the two-dimensional data of the preliminary massage point position and the proportion coefficient;
acquiring three-dimensional massage point position data of the target according to the two-dimensional data of the massage point position of the target and the point cloud data of the region to be massaged of the target object; and
generating a final massage track based on a preset massage track planning algorithm according to the three-dimensional massage point position data of the target;
wherein the preset massage track planning algorithm comprises grouping three-dimensional massage point positions of the target according to a preset grouping rule to obtain a plurality of massage point position groups;
generating a plurality of massage tracks according to the plurality of massage point position groups; and
splicing the plurality of segments of massage tracks to generate a final massage track.

In another aspect of the present application, it is preferable that the generating a final massage track based on a preset massage track planning algorithm according to the three-dimensional massage point position data of the target comprises:
selecting key points of massage point positions in a plurality of massage point position groups respectively;
respectively calculating node vectors of a plurality of massage point position groups based on the key points according to a preset first calculation method;
presetting boundary conditions of a plurality of massage point position groups;
according to the node vector and the preset boundary condition, respectively calculating control vertices of the plurality of massage point position groups according to a preset second calculation method;
generating a plurality of massage tracks according to the control vertices of the plurality of massage point position groups according to a preset third calculation method; and
splicing the plurality of segments of massage tracks to generate a preliminary massage track.

In another aspect of the present application, it is preferable that the generating a final massage track based on a preset massage track planning algorithm according to the three-dimensional massage point position data of the target further comprises:

according to the three-dimensional massage point position data of the target in the preliminary massage track, calculating the pose of each target three-dimensional massage point position according to a preset pose algorithm, and generating a final massage track.

In another aspect of the present application, preferably, the preset grouping rule comprises:
acquiring a quantity of massage point positions according to the three-dimensional massage point position data of the target;
presetting a minimum quantity of the grouping unit;
judging whether the quantity of massage point positions is greater than a preset minimum quantity of grouping units;
if the quantity of massage point positions is greater than a preset minimum quantity of grouping units, successively sliding a frame to take points for grouping according to the preset minimum quantity of grouping units until the quantity of massage point positions of the last group is less than or equal to the preset minimum quantity of grouping units; and
if the quantity of massage point positions is less than or equal to the preset minimum quantity of grouping units, no grouping is required.

In another aspect of the present application, it is preferable that the preset first calculation method includes:
setting the preset first-end node repetition degree to 4;

$${u}_{0} = {u}_{1} = {u}_{2} = {u}_{3} = 0$$

$${u}_{i + 3} = {u}_{i + 2} + \frac{\vert {p}_{i} - {p}_{i - 1} \vert}{{\sum }_{t = 1}^{n} \vert {p}_{t} - {p}_{t - 1} \vert}$$

$${u}_{n + 2} = {u}_{n + 3} = {u}_{n + 4} = {u}_{n + 5} = 1$$

wherein the node vector is represented as U=[u₀, u₁, . . . , uₙ₊₅]; n represents a quantity of key points; i represents a current node sequence quantity; pᵢ: an i^{th} key point; t: a sequence number of summation of all key points; and pₜ: a t^{th} key point.

In another aspect of the present application, it is preferable that the preset second calculation method includes:
obtaining a control vertex by calculating an inverse matrix of a following square matrix;

$$[ \begin{matrix}1 & & & & & & & \\ {a}_{2} & {b}_{2} & {c}_{2} & & & & & \\ & & … & … & … & & & \\ & & & … & … & … & & \\ & & & & … & … & … & \\ & & & & & {a}_{n - 1} & {b}_{n - 1} & {c}_{n - 1} \\ & & & & & & & 1\end{matrix} ] [ \begin{matrix}{d}_{1} \\ {d}_{2} \\ ⋮ \\ ⋮ \\ ⋮ \\ {d}_{n - 1} \\ {d}_{n}\end{matrix} ] = [ \begin{matrix}{e}_{1} \\ {e}_{2} \\ ⋮ \\ ⋮ \\ ⋮ \\ {e}_{n - 1} \\ {e}_{n}\end{matrix} ]$$

where dᵢ is a control vertex; and aᵢ, bᵢ, cᵢ, eᵢ are all parameters related to key points and node vectors.

In another aspect of the present application, it is preferable that the preset third calculation method includes:

$$p ( u ) = \frac{{\sum }_{i = 0}^{n} {ω}_{i} {d}_{i} {N}_{i , k} ( u )}{{\sum }_{i = 0}^{n} {ω}_{i} {N}_{i , k} ( u )}$$

where u represents a node parameter and an argument ∈[0, 1]; dᵢ represents a control vertex; k represents a quantity of times of curves; N_{i,k}(u) represents a k^{th} B-spline fundamental function; and ωᵢ represents a weight factor.

In another aspect of the present application, preferably, the B-spline fundamental function formulation comprises:

$${N}_{i , 0} ( u ) = \{ \begin{matrix}1 , & {u}_{i} \leq u < {u}_{i + 1} \\ 0 , & others\end{matrix}$$

$${N}_{i , k} ( u ) = \frac{u - {u}_{i}}{{u}_{i + k} - {u}_{i}} {N}_{i , k - 1} ( u ) + \frac{{u}_{i + k + 1} - u}{{u}_{i + k + 1} - {u}_{i + 1}} {N}_{i + 1 , k - 1} ( u )$$

setting

$$\frac{0}{0} = 0$$

where N_{i,k}(u) represents a k^{th} B-spline fundamental function; and u represents a node parameter.

In another aspect of the present application, preferably, the three-dimensional massage point position data of the target comprises a three-dimensional coordinate value and a Z-axis direction vector of the massage point position; the three-dimensional coordinate value comprises X-axis, Y-axis and Z-axis data; the preset pose algorithm includes:
acquiring an X-axis direction vector of each three-dimensional massage point position of the target according to data of each target three-dimensional massage point position in the preliminary massage track;
acquiring a Y-axis direction vector according to a cross product of the X-axis and Z-axis direction vectors of each target three-dimensional massage point positions;
correcting the Z-axis direction vector by using a three-axis pair-wise perpendicular manner according to the direction vectors of the X-axis and Y-axis; and
obtaining the pose of each target three-dimensional massage point position, and generating a final massage track.

In another aspect of the present application, preferably, a system for generating massage tracks includes:
a select module configured for acquiring two-dimensional data of preliminary massage point positions according to a selected massage mode;
an acquisition module configured for acquiring point cloud data of a region to be massaged of a target object;
a first calculation module configured for determining a proportion coefficient according to the two-dimensional data of the preliminary massage point position and the point cloud data of the region to be massaged of the target object, and determining the two-dimensional data of the massage point position of the target based on the two-dimensional data of the preliminary massage point position and the proportion coefficient;
a second calculation module configured for acquiring three-dimensional massage point position data of the target according to the two-dimensional data of the massage point position of the target and the point cloud data of the region to be massaged of the target object;
a third calculation module configured for generating a final massage track based on a preset massage track planning algorithm according to the three-dimensional massage point position data of the target;
wherein the preset massage track planning algorithm comprises grouping three-dimensional massage point positions of the target according to a preset grouping rule to obtain a plurality of massage point position groups;
generating a plurality of massage tracks according to the plurality of massage point position groups; and
splicing the plurality of segments of massage tracks to generate a final massage track.

The above technical solution of the present application has the following advantageous technical effects.

The present application may select a massage mode, and there are different two-dimensional data of preliminary massage point positions according to different massage modes so as to improve individual adaptability. According to the individual differences, the two-dimensional data of the massage point positions of the target are obtained to further improve the individual adaptability, and also provide a basis for further massage track planning. The preset massage track planning algorithm reduces the amount of calculation and the planned massage track is smooth, which is conducive to promotion.

### BRIEF DESCRIPTION OF THE DRAWINGS

FIG. 1 is an overall flow diagram of an example of the present application.

### DETAILED DESCRIPTION OF THE EMBODIMENTS

The objects, technical solution, and beneficial effects of the present application will become more apparent from the detailed description set forth below when taken in conjunction with the drawings. It should be understood that the description is intended for purposes of illustration only, and is not intended to limit the scope of the present application. Further, in the following description, descriptions of well-known structures and techniques are omitted to avoid unnecessarily obscuring the concepts of the present application.

Obviously, the described examples are some, but not all, examples of the present application. Based on the examples in the application, all other examples obtained by a person skilled in the art without involving any inventive effort are within the scope of protection of the application.

In the description of the present application, it should be noted that, the terms “first”, “second” and “third” are used solely for descriptive purposes and are not to be construed as indicating or implying relative importance.

Furthermore, the technical features involved in the various embodiments of the application described below may be combined with each other as long as they do not conflict with each other.

In the following description, numerous specific details are set forth, such as device structures, materials, dimensions, processing techniques and techniques, in order to provide a more clear understanding of the present application. However, it will be understood by those skilled in the art that the present application may be practiced without these specific details.

### Example 1

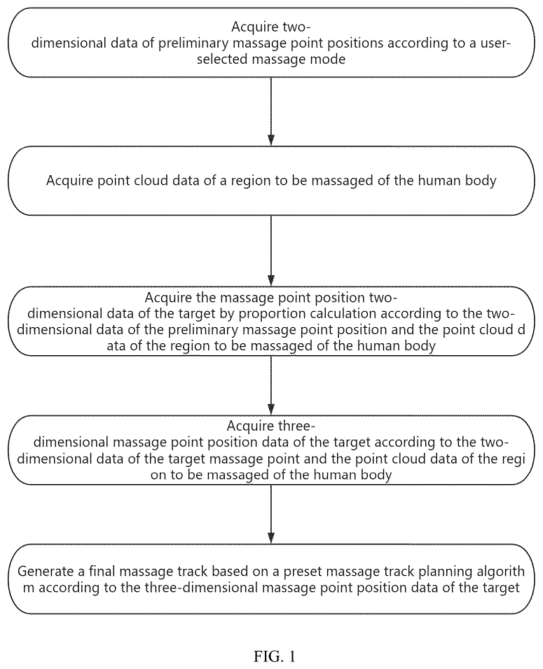

According to a method for generating massage tracks, as shown in FIG. 1, an overall flow chart of an example of the present application comprises the following steps.

Taking the human body massage as an example, two-dimensional data of preliminary massage point positions is acquired according to a selected massage mode. The method selected here can be a method selected by a user or recommended by large data. The specific content of the massage mode is not limited here, and may be for whole body, half-body, abdomen, or back. Alternatively, in the present example, the massage mode covers full-back massage point positions, a waist massage point, a shoulder massage point and an abdomen massage point, etc. and a standard massage scheme can be quickly formed. In addition, it also supports user-defined massage point positions, such as “Ashi point” in traditional Chinese medicine, with higher flexibility and adaptability. After selecting a massage mode, the two-dimensional data of the preliminary massage point position is correspondingly acquired in the corresponding massage mode.

The point cloud data of a region to be massaged of a target object is acquired. The specific method for acquiring the point cloud data of the region to be massaged of the target object is not limited herein. Optionally, a camera is used for photographing. The specific content of the camera is not limited herein, and the specific method for photographing is also not limited herein. Optionally, in the present example, the camera may be a depth camera, and the massage robot is controlled to move to a photographing pose for photographing. Optionally, in the present example, a person lies on or lies on a massage bed, and the massage robot is controlled to move above the target object for photographing. In the present example, the point cloud data of the region to be massaged of the target object may be directly acquired by using the depth camera for photographing.

A proportion coefficient is determined according to the two-dimensional data of the preliminary massage point position and the point cloud data of the region to be massaged of the target object, and the two-dimensional data of the massage point position of the target is determined based on the two-dimensional data of the preliminary massage point position and the proportion coefficient. The two-dimensional data of the preliminary massage point position is used as a reference, and the point cloud data of the region to be massaged of the target object is acquired according to an individual. A proportion coefficient is determined according to the two-dimensional data of the preliminary massage point position and the point cloud data of the region to be massaged of the target object. The two-dimensional data in the acquired point cloud data of the region to be massaged of the target object is used for calculation. For example, a person is fat, and the two-dimensional data of the preliminary massage point position can be expanded to obtain the target massage point position two-dimensional data. According to the change of the point cloud data, the density of the points taken of the massage point positions on the massage track is determined.

The three-dimensional massage point position data of the target is acquired according to the two-dimensional data of the massage point position of the target and the point cloud data of the region to be massaged of the target object. The specific content of the target three-dimensional massage point position data is not limited here. Optionally, in the present example, the target three-dimensional massage point position data comprises a three-dimensional coordinate value of the massage point position and a Z-axis direction vector, wherein the three-dimensional coordinate value comprises X-axis, Y-axis and Z-axis data. The target three-dimensional massage point position data is massage point position data adapted to the user himself. It is the basis for further planning the massage track.

A final massage track is generated based on a preset massage track planning algorithm according to the target three-dimensional massage point position data. The specific content of the preset massage track planning algorithm is not limited here, and a person skilled in the art would be able to understand that it works when the massage track planning can be performed. Optionally, in the present example, the generating a final massage track based on a preset massage track planning algorithm according to the three-dimensional massage point position data of the target comprises the followings.

The target three-dimensional massage point positions are grouped according to a preset grouping rule to obtain a plurality of massage point position groups; the specific content of the preset grouping rule is not defined herein, and may be an average score according to the quantity of target three-dimensional massage point positions, or may be an allocation according to the minimum quantity of units. A person skilled in the art would be able to understand that it works when the grouping can be completed. Alternatively, in the present example, the preset grouping rule comprises the followings.

A quantity of massage point positions is acquired according to the three-dimensional massage point position data of the target.

The minimum quantity of grouping units is preset. The specific quantity of the minimum quantity of units of grouping is not limited here. Optionally, in the present example, the preset minimum quantity of units of grouping is 7, here the preset minimum quantity of units of grouping is 7 because 7 is the minimum quantity of points for realizing smooth concatenation, and the quantity of points is the minimum, so as to ensure that the matrix inversion is the simplest within the range meeting the requirements to simplify the calculation.

It is judged whether the quantity of massage point positions is greater than a preset minimum quantity of grouping units. Here, firstly, it is judged whether the quantity of massage point positions is greater than 7.

If the quantity of massage point positions is greater than a preset minimum quantity of grouping units, successively sliding a frame to take points for grouping according to the preset minimum quantity of grouping units until the quantity of massage point positions of the last group is less than or equal to the preset minimum quantity of grouping units. Here, the specific contents of successively sliding the frame to take points for the grouping are a first group 1-7, a second group 2-8 and a third group 3-9, and so on successively. The quantity of points in each group is 7, the last group is determined by the quantity of points, and is not greater than 7 points.

If the quantity of massage point positions is less than or equal to the preset minimum quantity of grouping units, no grouping is required. The grouping can generate a plurality of massage tracks, and the plurality of massage tracks are spliced, and the plurality of massage track formed by the grouping can ensure a smooth transition at the splice.

The key points of massage point positions are selected in a plurality of massage point position groups respectively. Alternatively, in the present example, it may be the massage point positions that must be passed during the massage process. The quantity of key points is not limited herein. Alternatively, in the present example, the quantity of key points in each massage point position group is not greater than the quantity of massage point positions in the corresponding massage point position group.

The node vectors of a plurality of massage point position groups based on the key points are respectively calculated according to a preset first calculation method, wherein a first and a last node repetition degree is preset in the first calculation method. The preset first-end node repetition degree is not limited here. Optionally, in the present example, the preset first-end node repetition degree is 4. The repetition degree of 4 is to ensure that the first and last points of each massage point position group is at the key point, so that the repetition degree of the first and last nodes is set as (3+1). Optionally, in the present example, the preset first calculation method comprises: setting the preset first-end node repetition degree to 4;

$${u}_{0} = {u}_{1} = {u}_{2} = {u}_{3} = 0$$

$${u}_{i + 3} = {u}_{i + 2} + \frac{\vert {p}_{i} - {p}_{i - 1} \vert}{{\sum }_{t = 1}^{n} \vert {p}_{t} - {p}_{t - 1} \vert}$$

$${u}_{n + 2} = {u}_{n + 3} = {u}_{n + 4} = {u}_{n + 5} = 1$$

wherein the node vector is represented as U=[u₀, u₁, . . . , uₙ₊₅]; n represents a quantity of key points; i represents a current node sequence quantity; pᵢ: an i^{th} key point; t: a sequence number of summation of all key points; and pₜ: a t^{th} key point. The existence of repeated nodes can only provide n linear equations for n+2 unknown control vertices, so that two boundary conditions should be added to both ends of the curve to make the quantity of equations equal to the quantity of unknown control vertices. Thus, the control vertices can be solved;

The boundary conditions of a plurality of massage point position groups are preset. The specific contents of boundary conditions are not limited here. Optionally, the boundary conditions are a tangent vector condition, a free end point condition and a closed curve condition, etc. wherein the tangent vector condition requires that the tangential direction is fixed, the free end point condition is suitable for the case where the curvature of the first end point is zero, and the closed curve condition is suitable for the case where the first and last points are overlapped and are second-order continuous. Furthermore, in the present example, the free end point condition is used as the boundary condition to ensure that the continuity of the curve at the first end point is optimal.

According to the node vector and the preset boundary condition, the control vertices of the plurality of massage point position groups are respectively calculated according to a preset second calculation method. Optionally, in the present example, the preset second calculation method includes:
obtaining a control vertex by calculating an inverse matrix of a following square matrix;

$$[ \begin{matrix}1 & & & & & & & \\ {a}_{2} & {b}_{2} & {c}_{2} & & & & & \\ & & … & … & … & & & \\ & & & … & … & … & & \\ & & & & … & … & … & \\ & & & & & {a}_{n - 1} & {b}_{n - 1} & {c}_{n - 1} \\ & & & & & & & 1\end{matrix} ] [ \begin{matrix}{d}_{1} \\ {d}_{2} \\ ⋮ \\ ⋮ \\ ⋮ \\ {d}_{n - 1} \\ {d}_{n}\end{matrix} ] = [ \begin{matrix}{e}_{1} \\ {e}_{2} \\ ⋮ \\ ⋮ \\ ⋮ \\ {e}_{n - 1} \\ {e}_{n}\end{matrix} ]$$

where dᵢ is a control vertex; and aᵢ, bᵢ, cᵢ, eᵢ are all parameters related to key points and node vectors; the control top point can be obtained by matrix operation; and the inverse of the previous square matrix is required to find the control vertex. The calculation of the inverse matrix is very cumbersome. When the order is higher, the calculated amount of the inverse matrix doubles. Therefore, with regard to the track planning of the massage point positions with too many points in the present example, it is necessary to perform grouping processing in advance, respectively perform massage track planning on each group of points, and finally perform multi-segment massage track splicing to obtain a smooth massage track.
generating a plurality of massage tracks according to the control vertices of the plurality of massage point position groups according to a preset third calculation method. The specific content of the preset third calculation method is not limited here. Optionally, in the present example, the preset third calculation method comprises generating a massage track according to a node vector, a control vertex and a weight factor;

$$p ( u ) = \frac{{\sum }_{i = 0}^{n} {ω}_{i} {d}_{i} {N}_{i , k} ( u )}{{\sum }_{i = 0}^{n} {ω}_{i} {N}_{i , k} ( u )}$$

where u represents a node parameter and an argument ∈[0, 1]; dᵢ represents a control vertex; k represents a quantity of times of curves; N_{i,k}(u) represents a k^{th} B-spline fundamental function; and ωᵢ represents a weight factor; wherein the B-spline fundamental function formulation comprises:

$${N}_{i , 0} ( u ) = \{ \begin{matrix}1 , & {u}_{i} \leq u < {u}_{i + 1} \\ 0 , & others\end{matrix}$$

$${N}_{i , k} ( u ) = \frac{u - {u}_{i}}{{u}_{i + k} - {u}_{i}} {N}_{i , k - 1} ( u ) + \frac{{u}_{i + k + 1} - u}{{u}_{i + k + 1} - {u}_{i + 1}} {N}_{i + 1 , k - 1} ( u )$$

setting

$$\frac{0}{0} = 0$$

where N_{i,k}(u) represents a k^{th} B-spline fundamental function; and u represents a node parameter;
splicing the plurality of segments of massage tracks to generate a preliminary massage track.

In an example of the present application, further, the generating a final massage track based on a preset massage track planning algorithm according to the three-dimensional massage point position data of the target further includes:
according to the three-dimensional massage point position data of the target in the preliminary massage track, calculating the pose of each target three-dimensional massage point position according to a preset pose algorithm, and generating a final massage track. Furthermore, optionally, in the present example, the three-dimensional massage point position data of the target comprises a three-dimensional coordinate value and a Z-axis direction vector of the massage point position; the three-dimensional coordinate value comprises X-axis, Y-axis and Z-axis data; the preset pose algorithm includes:
acquiring an X-axis direction vector of each three-dimensional massage point position of the target according to data of each target three-dimensional massage point position in the preliminary massage track;
acquiring a Y-axis direction vector according to a cross product of the X-axis and Z-axis direction vectors of each target three-dimensional massage point positions;
correcting the Z-axis direction vector by using a three-axis pair-wise perpendicular manner according to the direction vectors of the X-axis and Y-axis; and
obtaining the pose of each target three-dimensional massage point position, and generating a final massage track.

The present example can select a massage mode, comprising a self-defined mode, and having different two-dimensional data of preliminary massage point positions according to different massage modes so as to improve individual adaptability. The two-dimensional data of target massage point positions is obtained according to individual differences, further improving individual adaptability, and also providing a basis for further massage track planning. According to the preset massage track planning algorithm, the quantity of massage point positions are grouped, the grouping is made according to the minimum quantity of points for smooth splicing, and the quantity of points is the minimum, so as to ensure that the matrix inversion is the simplest within the range meeting the requirements to simplify the calculation. The multi-segment planning massage track, and then splicing are made to achieve smooth transition of massage tracks, and to save computing power to the greatest extent. It is conducive to promotion.

### Example 2

A system for generating massage tracks includes:
a select module configured for acquiring two-dimensional data of preliminary massage point positions according to a selected massage mode; here, the specific content of the massage mode is not limited here, and may be for whole body, half-body, abdomen, or back; alternatively, in the present example, the massage mode covers full-back massage point positions, a waist massage point, a shoulder massage point and an abdomen massage point, etc. and a standard massage scheme can be quickly formed; in addition, it also supports user-defined massage point positions, such as “Ashi point” in traditional Chinese medicine, with higher flexibility and adaptability; after selecting a massage mode, the two-dimensional data of the preliminary massage point position is correspondingly acquired in the corresponding massage mode;
an acquisition module configured for acquiring the point cloud data of a region to be massaged of a target object; here, the specific method for acquiring the point cloud data of the region to be massaged of the target object is not limited herein; optionally, a camera is used for photographing; the specific content of the camera is not limited herein, and the specific method for photographing is also not limited herein; optionally, in the present example, the camera may be a depth camera, and the massage robot is controlled to move to a photographing pose for photographing; optionally, in the present example, a person lies on or lies on a massage bed, and the massage robot is controlled to move above the target object for photographing; and in the present example, the point cloud data of the region to be massaged of the target object may be directly acquired by using the depth camera for photographing.
a first calculation module configured for determining a proportion coefficient according to the two-dimensional data of the preliminary massage point position and the point cloud data of the region to be massaged of the target object, and determining the two-dimensional data of the massage point position of the target based on the two-dimensional data of the preliminary massage point position and the proportion coefficient; the two-dimensional data of the preliminary massage point position is used as a reference, and the point cloud data of the region to be massaged of the target object is acquired according to an individual, and the two-dimensional data of the massage point position of the target is acquired by calculating according to a proportion; for example, a person is fat, and the two-dimensional data of the preliminary massage point position can be expanded to obtain the target massage point position two-dimensional data. According to the change of the point cloud data, the density of the points taken of the massage point positions on the massage track is determined;
a second calculation module configured for acquiring the three-dimensional massage point position data of the target according to the two-dimensional data of the massage point position of the target and the point cloud data of the region to be massaged of the target object; here, the specific content of the target three-dimensional massage point position data is not limited; optionally, in the present example, the target three-dimensional massage point position data comprises a three-dimensional coordinate value of the massage point position and a Z-axis direction vector, where the three-dimensional coordinate value comprises X-axis, Y-axis and Z-axis data; the target three-dimensional massage point position data is massage point position data adapted to the user himself, it is the basis for further planning the massage track; and
a third calculation module configured for generating a final massage track based on a preset massage track planning algorithm according to the three-dimensional massage point position data of the target. The specific content of the preset massage track planning algorithm is not limited here, and a person skilled in the art would be able to understand that it works when the massage track planning can be performed. Optionally, in the present example, this example comprises:
grouping three-dimensional massage point positions of the target according to a preset grouping rule to obtain a plurality of massage point position groups;
selecting key points of massage point positions in a plurality of massage point position groups respectively;
respectively calculating node vectors of a plurality of massage point position groups based on the key points according to a preset first calculation method, wherein a first and a last node repetition degree is preset in the first calculation method;
presetting boundary conditions of a plurality of massage point position groups;
according to the node vector and the preset boundary condition, respectively calculating control vertices of the plurality of massage point position groups according to a preset second calculation method;
generating a plurality of massage tracks according to the control vertices of the plurality of massage point position groups according to a preset third calculation method; and
splicing the plurality of segments of massage tracks to generate a preliminary massage track.

Furthermore, in the present example, the generating a final massage track based on a preset massage track planning algorithm according to the three-dimensional massage point position data of the target further comprises:
according to the three-dimensional massage point position data of the target in the preliminary massage track, calculating the pose of each target three-dimensional massage point position according to a preset pose algorithm, and generating a final massage track.

It should be understood that the above-described examples of the application are merely illustrative of or explanatory of the principles of the application, and are not restrictive of the application. Accordingly, it is intended to embrace all such alternatives, modifications, and variations that fall within the spirit and scope of the present application. Furthermore, it is intended that the appended claims cover all such variations and modifications as come within the scope and range of equivalents of the claims appended hereto.

The application has been described above with reference to examples thereof. However, these examples are for illustrative purposes only and are not intended to limit the scope of the present application. It is intended that the scope of the application be defined by the claims appended hereto and their equivalents. Various alternatives and modifications can be devised by those skilled in the art without departing from the scope of the application, and these alternatives and modifications are intended to be included within the scope of the application.

Although embodiments of the present application have been described in detail, it should be understood that various changes, substitutions and alterations can be made herein without departing from the spirit and scope of the application.

Obviously, the above-described embodiments are merely illustrative for clarity of the examples given and are not restrictive of the implementations. It will be apparent to those skilled in the art that various other modifications and variations can be made in the application without departing from the scope or spirit of the application. All implementations need not be, and cannot be, exhaustive. The obvious changes or alterations resulting therefrom are still within the scope of protection of the application.

Those skilled in the art will appreciate that embodiments of the present application may be provided as a method, a system, or a computer program product. Thus, the present application may take the form of an entire hardware embodiment, an entire software embodiment, or an embodiment combining software and hardware aspects. Moreover, the present application may take the form of a computer program product embodied on one or more computer-usable storage media (including, but not limited to, magnetic disk storage, CD-ROM, optical storage, and the like) having computer-usable program code embodied therein.

What is claimed is:

1. A method for generating massage tracks, comprising:
acquiring two-dimensional data of preliminary massage point positions according to a selected massage mode;
acquiring point cloud data of a region to be massaged of a target object;
determining a proportion coefficient according to the two-dimensional data of the preliminary massage point position and the point cloud data of the region to be massaged of the target object, and determining the two-dimensional data of the massage point position of the target based on the two-dimensional data of the preliminary massage point position and the proportion coefficient;
acquiring three-dimensional massage point position data of the target according to the two-dimensional data of the massage point position of the target and the point cloud data of the region to be massaged of the target object; and
generating a final massage track based on a preset massage track planning algorithm according to the three-dimensional massage point position data of the target; wherein the preset massage track planning algorithm comprises grouping three-dimensional massage point positions of the target according to a preset grouping rule to obtain a plurality of massage point position groups;
generating a plurality of massage tracks according to the plurality of massage point position groups; and
splicing the plurality of segments of massage tracks to generate a final massage track.

2. The method for generating massage tracks according to claim 1, wherein the generating a final massage track based on a preset massage track planning algorithm according to the three-dimensional massage point position data of the target comprises:
selecting key points of massage point positions in a plurality of massage point position groups respectively;
respectively calculating node vectors of a plurality of massage point position groups based on the key points according to a preset first calculation method;
presetting boundary conditions of a plurality of massage point position groups;
according to the node vector and the preset boundary condition, respectively calculating control vertices of the plurality of massage point position groups according to a preset second calculation method;
generating a plurality of massage tracks according to the control vertices of the plurality of massage point position groups according to a preset third calculation method; and
splicing the plurality of segments of massage tracks to generate a preliminary massage track.

3. The method for generating massage tracks according to claim 2, wherein the generating a final massage track based on a preset massage track planning algorithm according to the three-dimensional massage point position data of the target further comprises:
according to the three-dimensional massage point position data of the target in the preliminary massage track, calculating the pose of each target three-dimensional massage point position according to a preset pose algorithm, and generating a final massage track.

4. The method for generating massage tracks according to claim 2, wherein the preset grouping rule comprises:
acquiring a quantity of massage point positions according to the three-dimensional massage point position data of the target;
presetting a minimum quantity of the grouping unit;
judging whether the quantity of massage point positions is greater than a preset minimum quantity of grouping units;
if the quantity of massage point positions is greater than a preset minimum quantity of grouping units, successively sliding a frame to take points for grouping according to the preset minimum quantity of grouping units until the quantity of massage point positions of the last group is less than or equal to the preset minimum quantity of grouping units; and
if the quantity of massage point positions is less than or equal to the preset minimum quantity of grouping units, no grouping is required.

5. The method for generating massage tracks according to claim 2, wherein the preset first calculation method comprises:
setting the preset first-end node repetition degree to 4;
$${u}_{0} = {u}_{1} = {u}_{2} = {u}_{3} = 0$$
$${u}_{i + 3} = {u}_{i + 2} + \frac{\vert {p}_{i} - {p}_{i - 1} \vert}{{\sum }_{t = 1}^{n} \vert {p}_{t} - {p}_{t - 1} \vert}$$
$${u}_{n + 2} = {u}_{n + 3} = {u}_{n + 4} = {u}_{n + 5} = 1$$
wherein the node vector is represented as U=[u₀, u₁, . . . , uₙ₊₅]; n represents a quantity of key points; i represents a current node sequence quantity; pᵢ: an i^{th} key point; t: a sequence number of summation of all key points; and pₜ: a t^{th} key point.

6. The method for generating massage tracks according to claim 2, wherein the preset second calculation method comprises:
obtaining a control vertex by calculating an inverse matrix of a following square matrix;
$$[ \begin{matrix}1 & & & & & & & \\ {a}_{2} & {b}_{2} & {c}_{2} & & & & & \\ & & … & … & … & & & \\ & & & … & … & … & & \\ & & & & … & … & … & \\ & & & & & {a}_{n - 1} & {b}_{n - 1} & {c}_{n - 1} \\ & & & & & & & 1\end{matrix} ] [ \begin{matrix}{d}_{1} \\ {d}_{2} \\ ⋮ \\ ⋮ \\ ⋮ \\ {d}_{n - 1} \\ {d}_{n}\end{matrix} ] = [ \begin{matrix}{e}_{1} \\ {e}_{2} \\ ⋮ \\ ⋮ \\ ⋮ \\ {e}_{n - 1} \\ {e}_{n}\end{matrix} ]$$
wherein dᵢ is a control vertex; and aᵢ, bᵢ, cᵢ, eᵢ are all parameters related to key points and node vectors.

7. The method for generating massage tracks according to claim 2, wherein the preset third calculation method comprises:
$$p ( u ) = \frac{{\sum }_{i = 0}^{n} {ω}_{i} {d}_{i} {N}_{i , k} ( u )}{{\sum }_{i = 0}^{n} {ω}_{i} {N}_{i , k} ( u )}$$
wherein u represents a node parameter and an argument ∈[0, 1]; dᵢ represents a control vertex; k represents a quantity of times of curves; N_{i,k}(u) represents a k^{th} B-spline fundamental function; and ωᵢ represents a weight factor.

8. The method for generating massage tracks according to claim 7, wherein the B-spline fundamental function formulation comprises:
$${N}_{i , 0} ( u ) = \{ \begin{matrix}1 , & {u}_{i} \leq u < {u}_{i + 1} \\ 0 , & others\end{matrix}$$
$${N}_{i , k} ( u ) = \frac{u - {u}_{i}}{{u}_{i + k} - {u}_{i}} {N}_{i , k - 1} ( u ) + \frac{{u}_{i + k + 1} - u}{{u}_{i + k + 1} - {u}_{i + 1}} {N}_{i + 1 , k - 1} ( u )$$
setting
$$\frac{0}{0} = 0$$
wherein N_{i,k}(u) represents a k^{th} B-spline fundamental function; and u represents a node parameter.

9. The method for generating massage tracks according to claim 3, wherein the three-dimensional massage point position data of the target comprises a three-dimensional coordinate value and a Z-axis direction vector of the massage point position; the three-dimensional coordinate value comprises X-axis, Y-axis and Z-axis data; the preset pose algorithm comprises:
acquiring an X-axis direction vector of each three-dimensional massage point position of the target according to data of each target three-dimensional massage point position in the preliminary massage track;
acquiring a Y-axis direction vector according to a cross product of the X-axis and Z-axis direction vectors of each target three-dimensional massage point positions;
correcting the Z-axis direction vector by using a three-axis pair-wise perpendicular manner according to the direction vectors of the X-axis and Y-axis; and
obtaining the pose of each target three-dimensional massage point position, and generating a final massage track.

10. A system for generating massage tracks, comprising:
a select module configured for acquiring two-dimensional data of preliminary massage point positions according to a selected massage mode;
an acquisition module configured for acquiring point cloud data of a region to be massaged of a target object;
a first calculation module configured for determining a proportion coefficient according to the two-dimensional data of the preliminary massage point position and the point cloud data of the region to be massaged of the target object, and determining the two-dimensional data of the massage point position of the target based on the two-dimensional data of the preliminary massage point position and the proportion coefficient;
a second calculation module configured for acquiring three-dimensional massage point position data of the target according to the two-dimensional data of the massage point position of the target and the point cloud data of the region to be massaged of the target object;
a third calculation module configured for generating a final massage track based on a preset massage track planning algorithm according to the three-dimensional massage point position data of the target;
wherein the preset massage track planning algorithm comprises:
grouping three-dimensional massage point positions of the target according to a preset grouping rule to obtain a plurality of massage point position groups;
generating a plurality of massage tracks according to the plurality of massage point position groups; and
splicing the plurality of segments of massage tracks to generate a final massage track.
